(19) United States
(12) Patent Application Publication
(10) Pub. No.: US 2026/0224862 A1
(43) Pub. Date: Aug. 6, 2026

(54) BALLOON CATHETER AND METHOD FOR MANUFACTURING BALLOON CATHETER

(71) Applicant: TERUMO KABUSHIKI KAISHA, Tokyo (JP)

(72) Inventors: Hiroyuki KAYANUMA, Mishima Shizuoka (JP); Naoki KOINUMA, Fujinomiya Shizuoka (JP)

(21) Appl. No.: 19/575,762

(22) Filed: Mar. 23, 2026

Related U.S. Application Data
(63) Continuation of application No. PCT/JP2024/027066, filed on Jul. 30, 2024.

(30) Foreign Application Priority Data
Sep. 27, 2023 (JP) 2023-164697

(51) Int. Cl. A61M 25/10; A61M 25/00

(52) U.S. Cl. CPC A61M 25/1034; A61M 25/0097; A61M 2025/1004; A61M 2025/1061; A61M 2207/00

(57) ABSTRACT

A balloon catheter includes a shaft and a balloon surrounding a distal portion of the shaft and including: a distal fused portion fixed to the shaft, and a distal tapered portion positioned proximal to the distal fused portion. The distal tapered portion includes a first portion and a second portion that is positioned distal to the first portion, the first portion has a first wing curved in a circumferential direction of the shaft when the balloon is deflated, and the second portion does not have any wing.

### CROSS-REFERENCE TO RELATED APPLICATION

This application is a continuation of International Patent Application No. PCT/JP2024/027066 filed July 30, 2024, which is based upon and claims the benefit of priority from Japanese Patent Application No. 2023-164697, filed September 27, 2023, the entire contents of which are incorporated herein by reference.

### TECHNICAL FIELD

The present disclosure relates to a balloon catheter and a method for manufacturing the balloon catheter.

### BACKGROUND ART

In the medical field, balloon catheters are widely known for use in procedures for dilating a lesion (stenosis or the like) formed in a body lumen. The balloon catheter includes an elongated shaft portion and a radially inflatable balloon provided on a distal side of the shaft portion, and the deflated balloon can be advanced to reach a lesion through a narrow body lumen and then be inflated to widen the lesion.

There is a known method for manufacturing a balloon catheter. A balloon manufactured by the known method is arranged between a proximal sleeve and a distal sleeve by being folded, and a fold is formed in a region extending from the distal sleeve to the proximal sleeve.

### SUMMARY

For example, in procedures using a balloon catheter, such as pre-dilatation and post-dilatation of a single lesion, multi-vessel lesion treatment, and bifurcation lesion treatment, the balloon may be inflated and deflated a plurality of times such that it is inflated once and then deflated in a body lumen, moved to a lesion at a different position, and then inflated again.

However, when the balloon is repeatedly deformed from the folded state to the inflated state or from the inflated state to the folded state, it may be difficult for the balloon to be reduced in diameter to its pre-inflation profile when the balloon is deflated and folded.

In the conventional balloon catheter, the fold formed from a distal end to a proximal end of a distal tapered portion of the balloon in a longitudinal direction contributes to an improvement in re-wrapping properties, but this leads to a decrease in bending properties or flexibility in a direction orthogonal to the fold. Therefore, the conventional balloon catheter may have reduced operability when advancing inside a blood vessel.

At least one embodiment of the present disclosure provides a balloon catheter in which it is possible to reduce a profile during deflation and improve operability by achieving both re-wrapping properties and bending properties of a balloon, and a method for manufacturing the balloon catheter.

In one embodiment, a balloon catheter comprises: a shaft; and a balloon surrounding a distal portion of the shaft and including: a distal fused portion fixed to the shaft, and a distal tapered portion positioned proximal to the distal fused portion. The distal tapered portion includes a first portion and a second portion that is positioned distal to the first portion, the first portion has a first wing curved in a circumferential direction of the shaft when the balloon is deflated, and the second portion does not have any wing.

According to at least one embodiment of the present disclosure, since the balloon catheter includes the first part and the second part in the distal end tapered portion, both re-wrapping properties and bending properties are achieved, and passability due to profile reduction and high operability due to an improvement in bending properties can thus be realized. Accordingly, in a procedure in which an inflation/deflation operation is repeatedly performed, one balloon catheter according to the present invention can be used a plurality of times without device replacement, and this can help reduce the number of devices used during the procedure.

### BRIEF DESCRIPTION OF DRAWINGS

FIG. 1 is a schematic diagram of a balloon catheter according to an embodiment.

FIG. 2 is a schematic cross-sectional view of the periphery of a distal portion of the balloon catheter.

FIG. 3 is a schematic cross-sectional view of the periphery of a distal tapered portion of the balloon catheter.

FIG. 4A is a schematic transverse sectional view of a first portion formed in the distal tapered portion.

FIG. 4B is a schematic transverse sectional view of a second portion formed in the distal tapered portion.

FIG. 4C is a schematic transverse sectional view of a third portion formed in the distal tapered portion.

FIG. 5 is a flowchart showing a method for manufacturing the balloon catheter.

FIG. 6 is a schematic diagram showing a configuration example of a fusion device.

### DETAILED DESCRIPTION

Hereinafter, embodiments of the present disclosure will be described in detail with reference to the drawings. Embodiments described herein are shown to embody the technical idea of the present invention and do not limit the present invention. Other embodiments, examples, technical operations and the like that could be conceived by those skilled in the art without departing from the gist of the present invention are all included in the scope and gist of the present invention and included in the invention recited in claims and the scope of equivalents thereof.

Furthermore, for the purpose of illustration and for ease of comprehension, the scale, aspect ratio, shape, and the like in the drawings attached may be changed from actual dimensions as appropriate and shown schematically. However, it is noteworthy that the drawings are examples and do not limit the interpretation of the present invention.

Note that in the following description, ordinal numbers such as “first” and “second” are used for convenience and do not define any order unless otherwise specified.

In addition, in the present specification, a “distal side” refers to a distal side of a balloon catheter 100 on which a balloon 140 is arranged, and a “proximal side” refers to a proximal side of the balloon catheter 100 on which a hub 180 is arranged, as the “proximal side” of the balloon catheter 100. Unless otherwise specified, a “distal portion” represents a certain range including a distal end (the most distal end) and its periphery, and a “proximal portion” represents a certain range including a proximal end (the most proximal end) and its periphery.

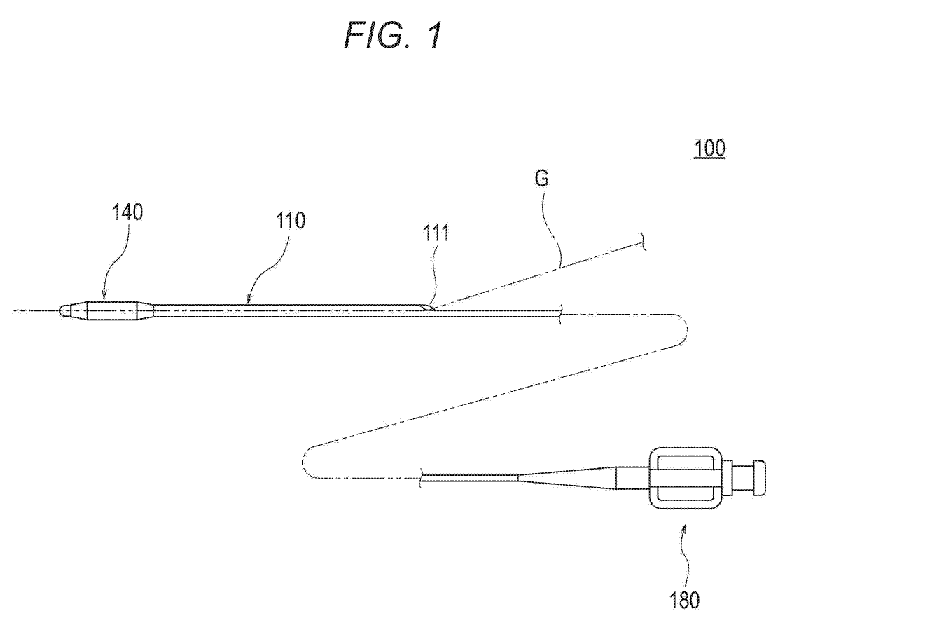

In FIG. 1, a “longitudinal direction” refers to a direction along a central axis of the balloon catheter 100. A “radial direction” is a direction away from or approaching a central axis in a cross-section orthogonal to the longitudinal axis with the central axis of the balloon catheter 100 as a reference axis. A “circumferential direction” is a rotation direction with the central axis of the balloon catheter 100 as a reference axis.

### Device Configuration

First, a configuration of the balloon catheter 100 according to the present embodiment will be described.

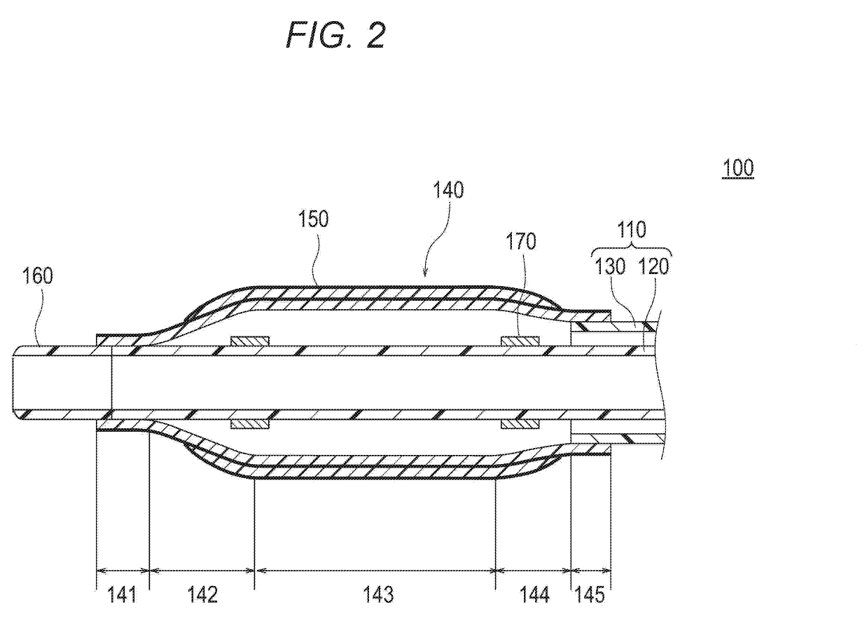

As shown in FIGS. 1 or 2 , the balloon catheter 100 is a medical device that widens a lesion such as a stenosis formed in a body lumen to perform treatment by inflating the balloon 140 arranged on a distal side of a shaft portion 110 within the lesion.

The balloon catheter 100 can be configured as, for example, a balloon catheter for PTCA dilation used to expand a stenosis of a coronary artery. However, the balloon catheter 100 can also be configured to be used for the purpose of treating and alleviating a stenosis formed in a biological organ such as another blood vessel, a bile duct, a trachea, an esophagus, another digestive tract, a urethra, an ear and nose lumen, or another organ.

The balloon catheter 100 is configured as a so-called “rapid exchange type catheter device” in which a guide wire port 111 from which a guide wire G is led out is provided near a distal portion side of the shaft portion 110. The balloon catheter 100 can also be configured as a so-called “over-the-wire type catheter device” in which a guide wire lumen 121 is formed to extend from the distal end to the proximal end of the shaft portion 110.

The shaft portion 110 includes an inner tube 120 in which the guide wire lumen 121 through which the guide wire G is inserted is formed, and an outer tube 130 forming a pressurizing medium lumen 131 between the outer tube 130 and the inner tube 120, through which a pressurizing medium can flow. The shaft portion 110 has a double tube structure in which the inner tube 120 is inserted into the outer tube 130 so that the inner tube 120 and the outer tube 130 are arranged concentrically.

Examples of the material constituting the inner tube 120 and the outer tube 130 include polyolefins such as polyethylene, polypropylene, ethylene-propylene copolymer, and ethylene-vinyl acetate copolymer, thermoplastic resins such as soft polyvinyl chloride, various rubbers such as silicone rubber and latex rubber, various elastomers such as polyurethane elastomers, polyamide elastomers, and polyester elastomers, and crystalline plastics such as polyamide, crystalline polyethylene, and crystalline polypropylene. In these materials, for example, antithrombotic substances such as heparin, prostaglandin, urokinase, and arginine derivatives can be blended to obtain a material having antithrombogenicity.

The balloon 140 is joined to a distal portion of the inner tube 120 in a liquid-tight and air-tight manner by laser welding. The balloon 140 has a distal portion joined to the inner tube 120 and a proximal portion joined to the outer tube 130.

The balloon 140 has an interior space into which a pressurizing medium can flow between the balloon 140 and the inner tube 120. The balloon 140 inflates when a pressurizing medium flows into the interior space. When the balloon 140 inflates, the balloon catheter 100 widens and dilates a stenosis formed in a body lumen by pushing a part of the balloon 140 against the stenosis.

As shown in FIG. 2, the balloon 140 has a distal fused portion 141 that is a certain region extending from the most distal end toward the proximal end of the balloon 140 and is fused to the inner tube 120, a distal tapered portion 142 arranged adjacent to a proximal end of the distal fused portion 141, a straight portion 143 arranged adjacent to a proximal end of the distal tapered portion 142, a proximal tapered portion 144 arranged adjacent to a proximal end of the straight portion 143, and a proximal fused portion 145 arranged adjacent to a proximal end of the proximal tapered portion 144 and fused to the outer tube 130.

The balloon 140 has a plurality of wings 150 formed to protrude outward in the radial direction, and the wing 150 is formed by imparting a folded shape having a fold 153 of a predetermined shape through a shaping process during manufacturing. The wing 150 is formed by folding the balloon 140 in the circumferential direction along the fold 153 formed at the base of the wing 150 during deflation. Here, the phrase “during deflation” means a deflated state immediately after the balloon 140 is taken out from a packaging container and before the pressurizing medium flows in during the procedure. Therefore, a deflated state when the balloon 140 is re-wrapped during the procedure is not included. The number, arrangement interval, cross-sectional shape (the shape of a transverse cross-section), and the like of the wings 150 are not particularly limited.

As a material constituting the balloon 140, for example, an organic polymer material can be used. Specifically, polymer materials such as polyolefin (for example, polyethylene, polypropylene, polybutene, ethylene-propylene copolymer, ethylene-vinyl acetate copolymer, ionomer, or mixtures of two or more kinds thereof), polyvinyl chloride, polyamide, polyamide elastomer, polyurethane, polyurethane elastomer, polyimide, or fluororesin, or mixtures thereof, or elastic resin materials of two or more of the polymer materials described above can be used. Among these, polyamide-based resins can be suitably used as a base material.

To the distal end of the inner tube 120, for example, a distal tip 160 can be attached to prevent a biological organ (an inner wall of a blood vessel or the like) from being damaged when the distal end of the balloon catheter 100 comes into contact with the biological organ. The distal tip 160 can be made of, for example, a more flexible resin material than the inner tube 120.

The inner tube 120 can be provided with contrast markers 170. The contrast markers 170 can be arranged at a position indicating a boundary with the distal side of the balloon 140 in the inner tube 120 and at a position indicating a boundary with the proximal side of the balloon 140 in the inner tube 120, for example.

As shown in FIG. 1, the hub 180 can be provided at a proximal portion of the shaft portion 110. The hub 180 can be connected to a supply device (not shown) such as an indeflator for supplying a pressurizing medium in a liquid-tight and air-tight manner.

The pressurizing medium (for example, saline or contrast agent) used for inflation of the balloon 140 can flow into the pressurizing medium lumen 131 of the shaft portion 110 via an internal space (lumen) of the hub 180. The pressurizing medium is supplied to an interior space of the balloon 140 through the pressurizing medium lumen 131.

In addition, the balloon 140 may have a coating formed to cover an outer surface thereof. The coating can be configured using, for example, a hydrophilic coating layer that improves slidability of the balloon 140 or a drug coating layer containing a predetermined drug. A specific material forming the hydrophilic coating layer or the drug coating layer is not particularly limited.

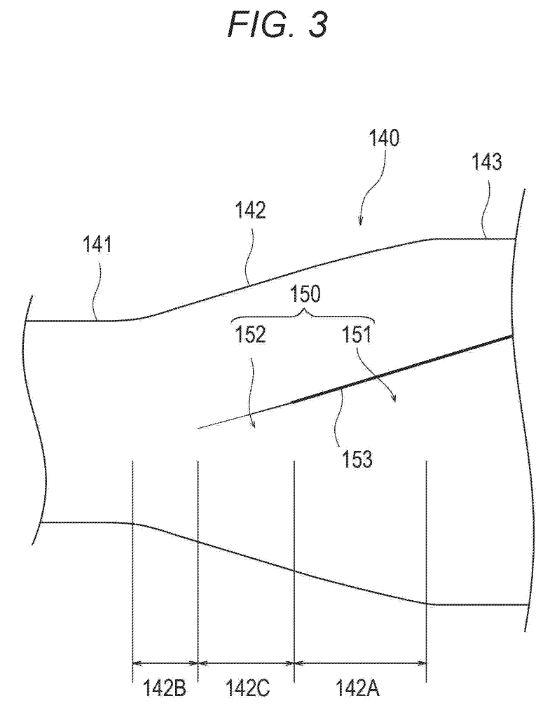

As shown in FIG. 3, the balloon 140 according to the present embodiment has four portions (first to third portions 142A to 142C), which are divided according to the state of the fold 153 imparted during shaping, in a region extending from the distal end to the proximal end of the distal tapered portion 142. Since the first to third portions 142A to 142C are positioned proximal to the distal fused portion 141, they are not fused to the inner tube 120.

As shown in FIG. 3, the distal tapered portion 142 includes the first portion 142A formed in a region extending from the proximal end toward the distal end, the second portion 142B positioned distal to the first portion 142A, and the third portion 142C formed between the first portion 142A and the second portion 142B. Since the balloon catheter 100 includes the distal tapered portion 142 in which the first to third portions 142A to 142C are formed, both re-wrapping properties and bending properties can be achieved.

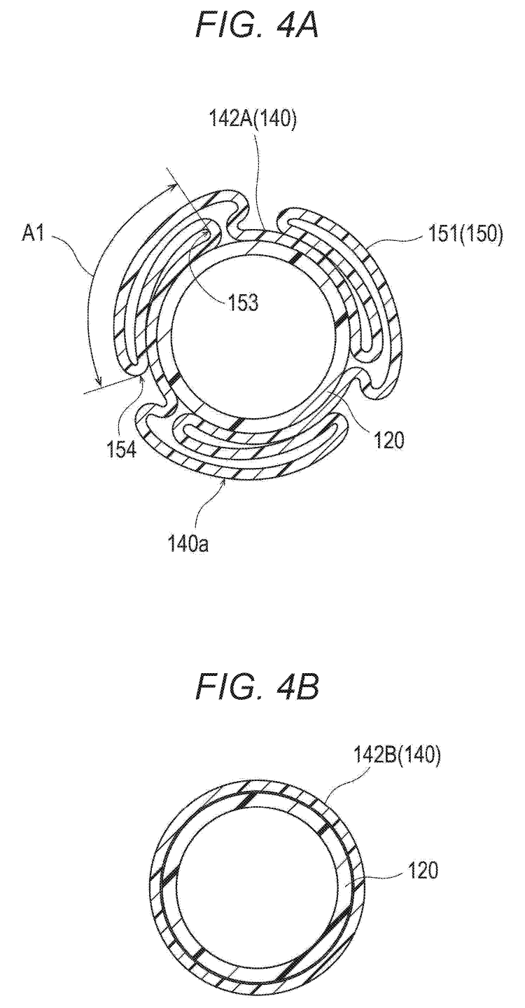

As shown in FIG. 3, the first portion 142A is a portion in which the wing 150 that is foldable during an inflation/deflation operation is formed in the distal tapered portion 142. In the present embodiment, the first portion 142A is formed in a predetermined region extending from the proximal end toward the distal end of the distal tapered portion 142. As shown in FIG. 4A, the first portion 142A has a first wing 151 functioning as the wing 150. The first wing 151 has a wing length A1 that is a length from the fold 153 to an apex portion 154 of the first wing 151. In the first portion 142A, the first wing 151 is folded along the fold 153 during deflation, and thus this contributes to an improvement in re-wrapping properties of the balloon catheter 100. As shown in FIG. 4A, an “outer peripheral surface 140a” is a surface of the distal tapered portion 142 facing radially outward in a state in which the balloon 140 is deflated and the wing 150 is folded in the circumferential direction.

As shown in FIG. 3, the second portion 142B is a portion that is positioned distal to the first portion 142A and does not have the wing 150 that is foldable during an inflation/deflation operation in the distal tapered portion 142. In the present embodiment, the second portion 142B is formed in a predetermined region extending from the distal end toward the proximal end of the distal tapered portion 142. As shown in FIG. 4B, the second portion 142B does not have the fold 153 acting when the wing 150 is folded. In the distal tapered portion 142, re-wrapping properties are improved due to the first portion 142A, but bending properties in a direction intersecting the longitudinal direction of the fold 153 may be reduced. Regarding this, the second portion 142B has higher bending properties in a direction intersecting the longitudinal direction than the first portion 142A since it does not have the fold 153. Therefore, the second portion 142B contributes to an improvement in operability of the balloon catheter 100 in a blood vessel.

As shown in FIG. 3, the third portion 142C is positioned between the first portion 142A and the second portion 142B, and includes a second wing 152 functioning as the wing 150. The second wing 152 has a wing length A2 that is a length from the fold 153 to an apex portion 155 of the second wing 152. The wing length A2 of the second wing 152 is shorter than the wing length A1 of the first wing 151.

In the present embodiment, as shown in FIG. 3, the third portion 142C is arranged so that a distal end is adjacent to a proximal end of the second portion 142B and a proximal end is adjacent to a distal end of the first portion 142A. The third portion 142C is a region where a transition from “having no wing 150” to “having a wing 150” occurs between the second portion 142B and the first portion 142A in the distal end tapered portion 142.

Since the third portion 142C has the length A2 shorter than the wing length A1 of the first wing 151 formed in the first portion 142A, its bending properties in a direction intersecting the longitudinal direction are higher than those of the first portion 142A, and are slightly lower than those of the second portion 142B that does not have the fold 153.

As described above, in the distal tapered portion 142, the second portion 142B that does not have the wing 150 is formed distal to the first portion 142A in which the wing 150 (first wing 151) is formed. In the balloon catheter 100, while the re-wrapping properties during an inflation/deflation operation are ensured by the first portion 142A, the bending properties on the distal side of the distal tapered portion 142 are increased by the second portion 142B and the operability in a blood vessel is thus increased.

In addition, the balloon catheter 100 has the third portion 142C between the first portion 142A and the second portion 142B in which the second wing 152 having the wing length A2 shorter than the wing length A1 of the first wing 151 formed in the first portion 142A is formed. The bending properties of the third portion 142C in a direction intersecting the longitudinal direction are higher than the bending properties of the first portion 142A. Therefore, in the balloon catheter 100, while the re-wrapping properties during an inflation/deflation operation are ensured by the first portion 142A and the third portion 142C, the bending properties on the distal side of the distal tapered portion 142 are further increased by the second 142B and the operability in a blood vessel can also be ensured. In addition, the third portion 142C has the second wing 152 having the wing length A2 shorter than the wing length A1 of the first wing 151 formed in the first portion 142A. Therefore, the third portion 142C can contribute to improving the bending properties, although its effect is less than that of the second portion 142B.

In the balloon 140, from the viewpoint of blood vessel passability due to profile reduction, the length in the longitudinal direction of the second portion 142B is preferably shorter than the length in the longitudinal direction of the distal fused portion 141.

In the balloon 140, from the viewpoint of blood vessel passability due to profile reduction, a ratio between a film thickness of the straight portion 143 continuous with the proximal end of the distal tapered portion 142 and a film thickness of the distal tapered portion 142 is preferably 1:1.10 or more and 1:1.18 or less, and more preferably 1:1.10 or more and less than 1:1.13.

### Manufacturing Method

Next, a method for manufacturing the balloon catheter 100 according to the present embodiment will be described.

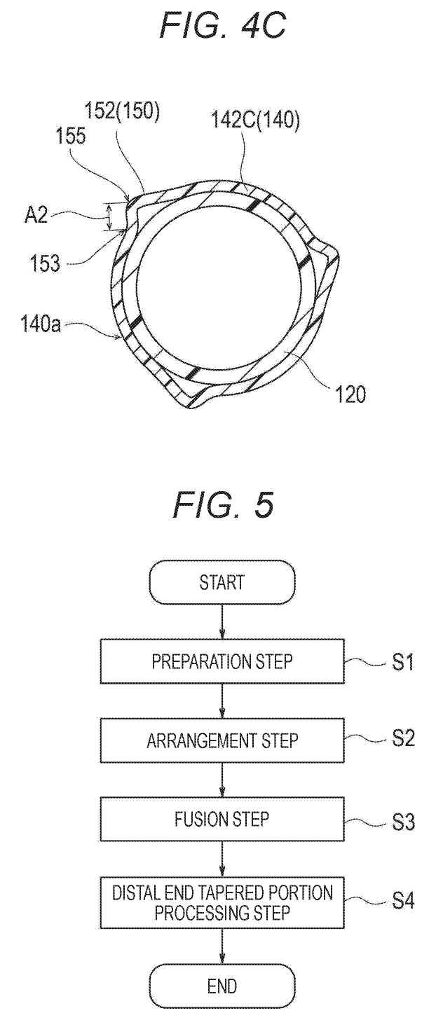

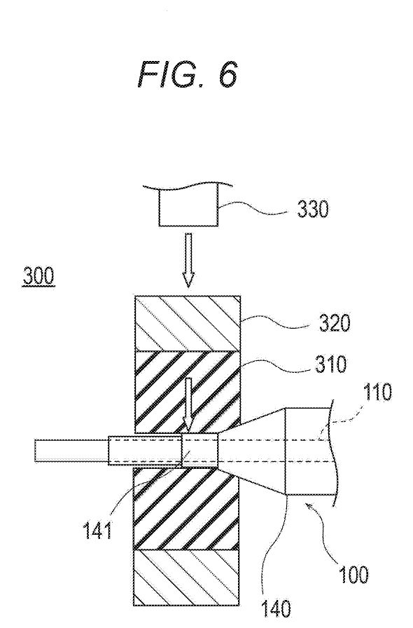

As shown in FIG. 5, the method for manufacturing the balloon catheter 100 includes the steps of: preparing the shaft portion 110 and the balloon 140 in which the wing 150 is formed in the distal tapered portion 142 positioned at the proximal end of the distal fused portion 141 fused to the shaft portion 110 (preparation step S1); arranging the balloon 140 on the distal side of the shaft portion 110 (arrangement step S2); fusing the balloon 140 to the shaft portion 110 by applying heat to the distal side of the balloon 140 to form the distal fused portion 141 (fusion step S3); and forming, by the heat applied to the distal side of the balloon 140, the second portion 142B in which the wing 150 that is foldable during an inflation/deflation operation of the balloon 140 is not formed, the second portion 142B being positioned distal to the first portion 142A in which the wing 150 that is foldable during an inflation/deflation operation of the balloon 140 is formed (distal tapered portion processing step S4).

The method for manufacturing the balloon catheter 100 may include other steps before and after each of the steps described above. In addition, the present manufacturing method presents the elements of the steps in an exemplary order, and is not limited to the order of the operations shown in FIG. 5. The order may be appropriately changed in consideration of workability and the like as long as the processing results do not contradict each other.

The present embodiment discloses a method for shaping the balloon 140 alone without joining the balloon 140 to the shaft portion 110. However, in the balloon shaping method, the balloon 140 can be shaped in a state in which it is joined to the shaft portion 110.

### Preparation Step

The preparation step S1 is a step of preparing the inner tube 120 and the outer tube 130 forming the shaft portion 110, and the balloon 140 to be fused to the shaft portion 110.

The balloon 140 prepared in the preparation step S1 is shaped to have the wing 150 in the balloon 140 using a shaping device. The shaping device includes a mold in which the wing 150 can be formed, and performs predetermined shaping by sandwiching a portion of the balloon 140.

### Arrangement Step

The arrangement step S2 is a step of arranging the shaped balloon 140 at a position where the shaft portion 110 is attached. In this case, the distal tip 160 may be arranged on the distal side of the balloon 140. In the arrangement step S2, the balloon 140, the shaft portion 110, and the like may be arranged in a fusion device 300, or the balloon 140 may be set in the fusion device in a state of being arranged on the shaft portion 110 in advance.

### Fusion Step

The fusion step S3 is a step of applying heat to the distal side of the balloon 140 to fuse the balloon 140 to the shaft portion 110, thereby forming the distal fused portion 141.

In the fusion step S3, the distal fused portion 141 is formed using the fusion device 300 as shown in FIG. 7. The fusion device 300 includes a support member 310, an external force applying member 320, and a laser irradiation portion 330.

The support member 310 is arranged to cover the shaft portion 110 and the balloon 140 in a state in which the deflated balloon 140 is arranged on the shaft portion 110. The support member 310 is made of a material that can be elastically deformed in the radial direction when subjected to an external force in order to prevent positional displacement of the balloon 140 relative to the shaft portion 110. The support member 310 supports the shaft portion 110 and the balloon 140 due to a radially inward force applied thereto.

The external force applying member 320 is arranged to cover a peripheral surface of the support member 310, and applies an external force to the support member 310. The external force applying member 320 is made of a material harder than the support member 310, and applies a radially inward external force to the support member 310 in a state of surrounding the support member 310. Therefore, the support member 310 is held in a state of supporting the shaft portion 110 and the balloon 140.

The laser irradiation portion 330 irradiates a fused portion on the distal side of the balloon 140 supported by the support member 310 with a laser of a predetermined wavelength to form the distal fused portion 141. As a laser irradiation range of the laser irradiation portion 330, at least a region where the distal fused portion 141 can be formed is irradiated with a laser.

Since the laser emitted from the laser irradiation portion 330 is emitted to the fused portion in a pinpoint manner, the range of thermal conduction is more localized than in conventional processing methods using a shrink tube. Therefore, it is possible to shorten the length in the longitudinal direction of the second portion 142B formed in the distal tapered portion 142. Therefore, the balloon catheter 100 can achieve both bending properties and re-wrapping properties in the distal tapered portion 142.

### Distal Tapered Portion Processing Step

The distal tapered portion processing step S4 is a step of forming the first to third portions 142A to 142C in the distal tapered portion 142 using the heat generated by the laser emitted during the formation of the distal fused portion 141.

In the distal tapered portion processing step S4, the heat generated by laser irradiation is transmitted to a predetermined region extending from the distal end to the proximal end of the distal tapered portion 142, whereby the second portion 142B is formed distal to the first portion 142A. In the second portion 142B and the third portion 142C, a portion of the distal tapered portion 142 melts and deforms due to the heat generated by laser irradiation, whereby the fold 153 disappears or partially deforms. In the distal tapered portion processing step S4, the heat generated by laser irradiation is not transmitted to the first portion 142A since the laser irradiation range is limited. Therefore, the fold 153 in the first portion 142A does not change from the state at the time of shaping, and the first wing 151 also maintains its shape.

Since the second portion 142B is a portion closest to the distal fused portion 141, the heat generated by laser irradiation is easily transmitted thereto, and the second portion 142B is formed in a state in which the wing 150 and the fold 153 disappear due to deformation by melting. Alternatively, in a case where the wing 150 is not formed in the shaping step, the second portion 142B is formed as a portion having no wing 150 regardless of the influence of the heat generated by laser irradiation.

The third portion 142C corresponds to a portion subjected to shaping in the shaping step. The fold 153 undergoes deformation by melting due to the heat generated by laser irradiation, whereby the second wing 152 is formed as shown in FIG. 4C.

Then, after the distal tapered portion processing step S4, the balloon catheter 100 can be manufactured by attaching the hub 180 to the shaft portion 110 and performing a processing step as necessary.

As described above, the balloon catheter 100 according to the present embodiment has: the shaft portion 110 having a hollow shape; and the deflatable balloon 140 arranged radially outward of the shaft portion 110, the balloon 140 has the distal fused portion 141 fixed to the shaft portion 110 and the distal tapered portion 142 positioned at the proximal end of the distal fused portion 141, the distal tapered portion 142 has the first portion 142A and the second portion 142B positioned distal to the first portion 142A, the first portion 142A has the first wing 151 curved in the circumferential direction in a deflated state of the balloon 140, and the second portion 142B does not have the wing 150.

In addition, the method for manufacturing the balloon catheter according to the present embodiment includes the steps of: preparing the shaft portion 110 and the balloon 140 in which a folded shape is imparted to the distal tapered portion 142 positioned at the proximal end of the distal fused portion 141 fused to the shaft portion 110; arranging the balloon 140 on the distal side of the shaft portion 110; applying heat to the distal side of the balloon 140 to fuse the balloon 140 to the shaft portion 110, thereby forming the distal fused portion 141; and forming, by the heat applied to the distal side of the balloon 140, the second portion 142B in which the folded shape that is foldable during an inflation/deflation operation of the balloon 140 is not imparted, the second portion 142B being positioned distal to the distal side than the first portion 142A in which the folded shape that is foldable during an inflation/deflation operation of the balloon 140 is imparted.

In the balloon catheter 100, the distal tapered portion 142 is formed with the first portion 142A having the folded shape imparted thereto and the second portion 142B having no folded shape imparted thereto. Therefore, in the balloon catheter 100, both re-wrapping properties and bending properties are achieved, and passability due to profile reduction and high operability due to an improvement in bending properties are thus realized. Accordingly, in a procedure in which an inflation/deflation operation is repeatedly performed, one balloon catheter 100 can be used a plurality of times without device replacement, and this can help reduce the number of devices used during the procedure.

What is claimed is:

1. A balloon catheter comprising:
a shaft; and
a balloon surrounding a distal portion of the shaft and including:
a distal fused portion fixed to the shaft, and
a distal tapered portion positioned proximal to the distal fused portion, wherein
the distal tapered portion includes a first portion and a second portion that is positioned distal to the first portion,
the first portion has a first wing curved in a circumferential direction of the shaft when the balloon is deflated, and
the second portion does not have any wing.

2. The balloon catheter according to claim 1, wherein
the distal tapered portion includes a third portion between the first and second portions,
the third portion has a second wing curved in the circumferential direction of the shaft when the balloon is deflated, and
in the circumferential direction, a length between a fold of the second wing and an apex of the second wing is shorter than a length between a fold of the first wing and an apex of the first wing.

3. The balloon catheter according to claim 1, wherein in a longitudinal direction of the shaft, a length of the second portion is shorter than a length of the distal fused portion.

4. The balloon catheter according to claim 1, wherein
the balloon has a straight portion continuous with a proximal end of the distal tapered portion, and
a ratio between a film thickness of the straight portion and a film thickness of the distal tapered portion is 1:1.10 or more and 1:1.18 or less.

5. The balloon catheter according to claim 4, wherein the ratio between the film thickness of the straight portion and the film thickness of the distal tapered portion is 1:1.10 or more and less than 1:1.13.

6. The balloon catheter according to claim 1, wherein the shaft includes an inner tube through which a guide wire is inserted and an outer tube arranged concentrically around the inner tube such that a pressurizing medium can flow between the inner and outer tubes.

7. The balloon catheter according to claim 6, wherein the distal fused portion is fixed to a distal portion of the inner tube, and the balloon further includes a proximal fused portion fixed to the outer tube.

8. The balloon catheter according to claim 6, wherein the shaft includes a guide wire port provided on a distal side of the shaft and through which the guide wire is led out.

9. The balloon catheter according to claim 6, further comprising:
a contrast marker provided on the inner tube and positioned radially inward of the first wing.

10. The balloon catheter according to claim 1, further comprising:
a hub provided at a proximal portion of the shaft, wherein
the shaft includes a lumen, and
the hub includes an internal space for supplying a pressurizing medium into the lumen of the shaft.

11. A balloon catheter comprising:
a shaft; and
a balloon made of a film, surrounding a distal portion of the shaft, and fixed to the shaft at distal and proximal ends of the balloon, wherein
the balloon includes, at a location different from the distal and proximal ends:
a first portion at which the film is folded in a circumferential direction of the shaft, and
a second portion distal to the first portion and free of folds.

12. The balloon catheter according to claim 11, wherein the balloon includes a third portion between the first and second portions and at which the film protrudes toward a radial direction of the shaft.

13. The balloon catheter according to claim 12, wherein
the film at the first portion includes a first fold and a first apex,
the film at the third portion includes a second fold and a second apex, and
in the circumferential direction, a length between the second fold and the second apex is shorter than a length between the first fold and the first apex.

14. The balloon catheter according to claim 11, wherein
the balloon includes a distal fused portion at the distal end, and
in a longitudinal direction of the shaft, a length of the second portion is shorter than a length of the distal fused portion.

15. The balloon catheter according to claim 11, wherein
the balloon includes:
a distal tapered portion including the first and second portions, and
a straight portion continuous with a proximal end of the distal tapered portion, and
a ratio between a film thickness of the straight portion and a film thickness of the distal tapered portion is 1:1.10 or more and 1:1.18 or less.

16. The balloon catheter according to claim 15, wherein the ratio between the film thickness of the straight portion and the film thickness of the distal tapered portion is 1:1.10 or more and less than 1:1.13.

17. The balloon catheter according to claim 11, wherein the shaft includes an inner tube through which a guide wire is inserted and an outer tube arranged concentrically around the inner tube such that a pressurizing medium can flow between the inner and outer tubes.

18. The balloon catheter according to claim 17, wherein the distal end of the balloon is fixed to a distal portion of the inner tube, and the proximal end of the balloon is fixed to the outer tube.

19. A method for manufacturing a balloon catheter, the method comprising:
preparing a shaft and a balloon having a wing that is folded in a circumferential direction;
arranging the balloon on a distal side of the shaft;
applying heat to a distal side of the balloon to fuse a distal portion of the balloon to the shaft; and
forming, by the heat applied to the distal side of the balloon, a portion proximal to the distal portion of the balloon and at which the wing no longer exists.

20. The method according to claim 19, wherein forming the portion proximal to the distal portion includes forming another portion proximal to the portion proximal to the distal portion of the balloon and at which the wing remains with a deformed fold.
